(19) United States
(12) Patent Application Publication
(10) Pub. No.: US 2013/0005048 A1
(43) Pub. Date: Jan. 3, 2013

(54) INDICATOR DEVICE

(76) Inventors: Monika Felten, Elkhart, IN (US); Juli Patricia Hood, Osceola, IN (US)

(21) Appl. No.: 13/172,307

(22) Filed: Jun. 29, 2011

(51) Int. Cl. G01N 21/78; C09B 62/00; C08G 63/91; C07K 14/435

(52) U.S. Cl. 436/164; 530/360; 534/617; 525/54.1; 525/54.21

(57) ABSTRACT

The present invention provides a test device for indicating the presence, concentration and effectiveness of chemical and biological agents. The test device interacts with the target agent producing a detectable signal indicating the presence, concentration and effectiveness of the agent.

### BACKGROUND OF THE INVENTION

Many processes and systems utilize the selective use of chemical or biological agents to achieve a desired result. Often, it is difficult or impossible to determine from visual observation whether the agent functioned as intended. As a result, a test device, or indicator, is often employed to provide evidence that the agent was present and functioned as intended. One common way to test for the presence of a chemical or biological agent is to use a test device that will react in the presence of the target agent in such a way that will provide a detectable signal.

An ideal test device will provide a positive signal in the presence of the targeted chemical or biological agent, and will provide a negative signal, or no signal, in the presence of all other agents or in the presence of no agents at all. Ideally, the difference between the positive and negative signals should be readily discernable.

One area in which the detection of chemical or biological agents is of particular utility is the health care industry. For example, hospitals, doctor's offices, and other medical care providers must ensure that all medical instruments are properly cleaned and sanitized to inhibit the spread of infection. Cleaning and sanitization are especially important for reusable medical devices so as to prevent the transmission of disease from patient to patient. The common practice in the industry is to use a two-stage process of first cleaning residue from a medical instrument, followed by sanitizing the instrument. The sanitization step will be ineffective if residue remains on the instrument following the cleaning step. The cleaning step generally involves using a specially configured medical instrument washer. As used herein, medical instrument washers (washers, or instrument washers), refers to a device, machine or process as is known in the art for cleaning and/or sanitizing medical instruments following use. Such medical instrument washers include, but are not limited to, specially adapted commercial dishwashers and ultrasonic cleaning systems. Typically, special detergents are used in medical instrument washers for breaking down soil-like materials—such as proteins, cellulose, fats, starches or sugars—as are found in blood and other tissues. Generally, medical instrument washer detergents include enzyme-based solutions, high-pH solutions, or a combination thereof. The instrument washer generally includes high-pressure water jets and elevated temperatures to help clean the medical instruments. In the typical configuration, the washer includes one or more programmable cycles, with each cleaning cycle including a combination of detergents, varying water temperatures, and water jets. The washer may include one or more water rinse, acid rinse, lubrication, or heated air drying cycles.

Medical instrument washer detergents typically function by breaking down soil-like materials which are found on soiled medical instruments. Breaking down the soil-like materials encourages their release from the medical instruments. These detergents typically act as hydrolyzing agents. Enzyme-based and high-pH solutions are examples of solutions capable of breaking down soil-like materials through a hydrolysis reaction.

Enzymes are proteins used to catalyze biological reactions. Enzymes enable one or more molecules, called substrates, to react and form one or more other molecules, called products. One such enzymatic reaction is the breaking down of substrates through hydrolysis. Enzymes are required to catalyze many of the processes required for biological cells to function properly. Enzymes can take many forms, and typically a given enzyme is only effective in serving as a catalyst for a single substrate or small class of substrates. As a result, a specific enzyme can be detected by monitoring for the transformation of a particular substrate. Since enzymes are substrate specific, testing for an enzyme will result in a negative indication in the presence of all but a specific enzyme. This property allows for detection of a specific enzyme in a solution containing many enzymes.

Enzyme-based detergents are one type of detergent used in medical instrument cleaners used to break down soil-like materials remaining on instruments following a medical event such as surgery. Enzymes are chosen which cleave or modify the soil-like materials that would typically be found on medical equipment. Breaking down the soil-like materials causes them to be more readily released from a medical instrument. For example, amylases and proteases are enzymes used in the digestive systems of animals to break down large molecules (starches and proteins, respectively), into smaller molecules to allow for easier digestion. These, and similar enzymes, are used in medical instrument washer detergents for breaking down starches, proteins or other soil-like materials found on surgical instruments.

High-pH, or basic, solutions are also effective when used as medical instrument washer detergents. Such solutions contain hydroxyl ions which aid in the hydrolysis of a soil-like material such as by attacking the carboxyl group of an ester or amide on the soil-like material. In this way, the soil-like material is cleaved into two or more parts, allowing for easier release of the soil-like material from medical instruments. Additionally, some detergents utilize surfactants which can help to dissolve or incorporate the soil-like materials into the detergent solution.

The standard practice in the industry for testing the effectiveness of the cleaning function of medical instrument washer detergents is to include a diagnostic plate in the washer, such as Tosi® by PEREG GmbH or Wash-Checks™ by SteriTec Products, Inc. These diagnostic plates are typically constructed from stainless steel to mimic surgical implements and are stained with a soil-like substances that mimics the properties of soil-like materials found on surgical equipment such that a proper washing cycle is indicated by a visually clean diagnostic plate.

Use of diagnostic plates presents at least three problems. First, the plates do not provide an indication that the detergent included proper active ingredients, such as enzymes or a high-pH solution. Since the plate is only evaluated based on a visual inspection, it is possible that unseen biological residue remains on the plate following cleaning. For example, in some cases, the action from the high-powered jets in the instrument washer physically removes the visible soil-like materials from the diagnostic plates, but leaves behind unseen materials. In such a case, the diagnostic plate would give the impression that the active ingredient in the detergent functioned properly when it did not. Second, the diagnostic plate does not provide conclusive evidence that the plates have been properly cleaned. As described above, the plate could emerge visually clean from the medical instrument washer, but for a reason other than proper concentration or effectiveness of the detergent. Third, the diagnostic plates are high in cost due to their construction from stainless steel. The high cost of the diagnostic plates may encourage minimal or sporadic monitoring of medical instrument washers which may lead to unseen biological agents remaining on medical instruments following cleaning which can lead to the spread of disease.

Other prior art systems use test strips for detecting the presence of enzymes in applications other than medical instrument washers. For example, U.S. Pat. No. 4,563,421 ('421, incorporated herein by reference) discloses a method for determining the presence of a target enzyme. The test strip of the '421 patent uses a starch chemically bonded to a pigment or dye, with the starch/dye complex dried onto a paper test strip. When an enzyme reacts with the starch, the starch is broken down and the dye is released from the test strip, causing a visible color change on the test strip. Thereby, a positive test result for the presence of a target enzyme is indicated by a color change on the test strip.

The drawback of the '421 patent, as applied to the use in medical instrument washers, is that false positive results could result if the test strip is contacted by a high-pressure water jet or ultrasonic action. Since the starch/dye complex of the '421 patent is only physically bound to the test strip through drying, rather than being chemically bonded, the starch/dye complex could be physically dislodged from the test strip during normal operation of the medical instrument washer which could result in a false positive test result, thereby being an ineffective solution to the present problem.

Other areas require analysis of the presence, concentration, or effectiveness of enzymatic or hydrolytic agents or surfactants. The test device of the present invention would be suitable in other applications where enzymes, hydrolytic agents or surfactants are used to break down substrates. For example, enzymes are used in various aspects of food production. One example is proteolytic cleavage of pepsin on various food products. In one example, pepsin will digest milk products leading to the curdling or coagulation of milk which makes it useful in the production of cheese. It is also used in other food applications like processing of soy or gelatin. Another example of proteolytic enzyme action in food applications is lactase. Lactase is a glycoside hydrolase enzyme that cleaves lactose into its constituent sugars, galactos and glucose. Lactase is used commercially to prepare lactose-free products, particularly milk, or in the preparation of ice cream, to make a creamier and sweeter-tasting product. Other enzymes used in many industrial applications are lipases. They are used to break down fats such as milk fat to give characteristic flavors to some cheeses, in production of personal care ingredients like isopropyl myristate, or isopropyl palmitate. These are but a few examples of areas where the analysis of the presence, concentration, and effectiveness of enzymatic, hydrolytic or surfactant agents is of utility.

Thus, an improved test device is needed for detecting the presence, concentration or effectiveness of chemical and biological agents.

### SUMMARY OF THE INVENTION

The present invention relates to the detection of chemical or biological agents. More specifically, the present invention relates to a test device which indicates the presence and/or concentration of enzymes, hydrolytic agents, or surfactants. In one specific application, the present invention relates to a test device, or indicator, for use in medical instrument washers for determining the presence, concentration and/or effectiveness of detergents used for cleaning medical instruments though the invention has wide applicability beyond medical instrument washers.

The test device of the present invention may be in the form of a test strip, and reacts in the presence of enzymatic, hydrolytic agents, or surfactants, resulting in a visible color change on the test device indicating the presence, concentration or effectiveness of such agents. The reactive portion of the test device includes a substrate, such as a protein, starch, sugar, cellulose, other soil-like materials, or a compound having a functional group which reacts—such as through a hydrolysis reaction—in the presence of such agents. The substrate is chemically bonded to a dye, thereby forming a substrate/dye compound. The substrate/dye compound is also chemically bonded to a carrier matrix which in turn may be affixed to a secondary carrier matrix such as a plastic strip to form the test device.

In one example of use, one or more test devices are placed in a medical instrument washer, and the washer is allowed to run for a washing cycle. After the washing cycle the devices(s) are analyzed for a change of color. A change of color on the test device indicates that the proper active ingredients were present in the detergent. The degree of color change on the test device correlates to the concentration of the active ingredients in the detergent solution.

The present invention is an improvement over the prior art since the present invention provides an indication of the presence of active ingredients in the target chemical or biological agent. Further, the present test devices are improvements on the prior art since they also indicate the relative concentration of the active ingredients in the detergent and are of lower cost.

The present invention is also an improvement upon existing strip-based detection methods since the present invention will withstand harsh environments such as temperatures or pressure which vary from ambient conditions, or environments which are subject to agitation or other disturbances. The present invention chemically bonds the substrate to a carrier matrix. This chemical bond between substrate and carrier helps ensure that the dye will only be removed from the test device in the presence of a target chemical or biological agent which facilitates a substrate reaction. High pressure water jets and other physical means will leave the dye in place, thereby preventing false positive results.

The present invention is also an improvement upon existing detection methods since the present invention may be modified to allow detection of both a first and a second chemical or biological agent. For example, the present invention may be configured to allow detection of an enzyme-based detergent in a first washer cycle of a medical instrument washer, and detection of an acid-based rinsing agent in a second washing cycle. In this way, the present invention provides superior diagnostic feedback as compared to either the strip-based or plate-based test devices of the prior art.

### BRIEF DESCRIPTION OF THE DRAWINGS

FIG. 1 is a perspective, exploded view of one embodiment of a test device.

FIG. 2 is a perspective view of the test device of FIG. 1 as assembled.

FIG. 3 is a perspective, exploded view of another embodiment of the test device.

FIG. 4 is a perspective view of the test device of FIG. 3 as assembled.

FIG. 5 is a perspective, exploded view of a further embodiment of the test device.

FIG. 6 is a perspective view of the test device of FIG. 5 as assembled.

### DESCRIPTION OF THE PREFERRED EMBODIMENTS

The present invention relates to test devices, or indicators, for indicating the presence and/or concentration of chemical or biological agents, such as enzymes, surfactants or hydrolytic agents. The test devices generally are defined as a carrier matrix bonded to a substrate which in turn is bonded to a dye, as described in detail below. In the presence of a target chemical or biological agent, the substrate releases the dye from the test device, causing a color change which indicates the presence of the agent. The test device as described herein has applicability in a variety of applications. One example of a suitable application is use in a medical instrument washer, though any reference to the same should not be read to limit the present invention.

The definitions and examples provided herein are meant to be illustrative of how various terms and phrases are understood within the context of the present invention, but should not be read to limit the scope of the invention.

Suitable substrates include compounds capable of reaction in the presence of target chemical and biological agents. Such substrates include, but are not limited to, proteins, cellulose, polysaccharides such as starches or sugars, lipids, and biological compounds found in tissue. Alternatively, the substrate is a compound having a functional group—such as a peptide or amino acid attached to a backbone such as polyethylene glycols or dextrans—which is susceptible to reaction in the presence of target chemical or biological agents; such a compound would function as a tissue-like substance, but could be synthesized from a non-biological source, as is known in the art. The substrate is suitable for coupling to a reactive dye and a carrier. To be suitable for coupling to a carrier, the substrate generally must have a reactive chemical group, or be capable of being modified to provide a reactive chemical group which allows covalent linking of the substrate to the dye and the carrier.

The test device of the present invention is designed to indicate the presence and/or concentration of target chemical or biological agents. Such agents include, but are not limited to, enzymes, hydrolytic agents and surfactants. Medical instrument washer detergents are but one example of a chemical or biological agent detectable by the present invention. One type of detergent includes one or more enzymes which are suitable for breaking down soil-like materials remaining on medical instruments following a medical procedure. This first detergent breaks down the soil-like materials at least through enzymatic cleavage. A second type of detergent is a high-pH, or basic detergent, which breaks down soil-like materials through hydrolysis. A third type of detergent includes a solution containing a combination of the first and second detergents. The test device of the present invention is suitable for indicating the effectiveness of each of these types of detergents. The test device of the present invention is also suitable to be used to indicate the effectiveness of enzyme-based, high-pH-based, or surfactant-based solutions as applied in applications other than medical instrument washers.

The test device of the present invention is adapted for use in medical instrument washers, though may be used in other applications which require indication of the presence of enzymatic, hydrolytic, surfactant or other similar chemical or biological agents. Such medical instrument washers are known in the art. One example of such a medical instrument washer is essentially a modified commercial dishwasher. Other washers use elements which emit ultrasonic waves to assist in the cleaning action. Additionally, the test device of the present invention is suitable for cases where medical instruments are manually cleaned. While the description herein describes use in a medical instrument washer, the test devices of the present invention may be adapted for use in a variety of applications where there is utility in the detection of chemical or biological agents.

Detectable enzymes include, but are not limited to, enzymes such as proteases and amylases that are present in detergents formulated for removing blood, starch, protein or other soil-like compounds found on surgical equipment. More specifically, subtilisin-type protease enzymes are readily detected using the test device described herein. Other detectable enzymes include amylase, cellulase or lipase. The test device described herein is readily adaptable to detect enzymes not specifically listed by choosing a substrate suitable for reaction in the presence of the target enzyme and bonding that substrate to a dye and a carrier. The test device may be adapted for detecting multiple enzymes, such as by bonding multiple substrates to a single carrier, or by adhering multiple carriers to a single secondary carrier.

Detectable high-pH solutions include chemical solutions which facilitate hydrolysis. As an example, such solutions include commercially-available medical instrument washer solutions. Such solutions have a high-pH and facilitate the hydrolysis of soil-like materials found in medical instrument washers.

The dyes suitable to be used in the present invention are organic compounds which can be covalently bonded to a substrate as is known in the art. Such reactive dyes include dyes suitable for bonding to a soil-like compound, such as mono- or dichloro-triazine compounds like Reactive Blue, triphenylmethane dyes such as Brilliant Blue or dyes modified to include reactive groups such as Vinylsulfone dyes and Bromo-acylamides, or similar reactive azo compounds, or combinations thereof, such as Reactive Orange 16. Reactive dyes are commercially available from chemical suppliers under brand names such as Cibacron®, Procion, Basilen, or Remazol®. Suitable substrate/dye compounds, such as Azocasein, are commercially available from Sigma-Aldrich® and similar chemical suppliers.

Other dyes suitable for use include dyes having a high affinity for proteinacous and cellulosic materials such as direct dyes having a reactive group suitable for bonding to a soil-like compound such as Congo Red, Direct Orange 31, Direct Blue 1, cationic monoazo dyes such as Chrysoidin, and other direct dyes as are known in the art. Direct dyes are dyes with good affinity to carriers, such as cotton and other cellulose or proteinacous materials, or silk, wool or nylon. As described in more detail below, direct dyes are generally adhered to the carrier as opposed to being coupled to a substrate. The direct dyes are generally used in conjunction with a dye/substrate, though they may be used independently. The direct dyes generally have indicator properties where they will exhibit a color change in particular reaction conditions. For example, Congo Red changes color based on the pH of the test solution. Other dyes have other indicator properties as are known in the art and may be used as a direct dye in the present invention.

The carrier is a matrix suitable for bonding to a substrate. Two types of carrier matrices are of particular applicability here, namely carboxyl-containing matrices and amine-containing matrices, as are known in the art. For example, paper or wood fibers are suitable cellulose-based carriers having a carboxyl-containing matrix. Other carriers, such as those fabricated from silk or wool, include proteins which have active amine groups, and are suitable carboxyl and amine-containing matrices. Similarly, paper, glass-fiber or membranes can be modified to include an active amine group by being treated with compounds such as polyamine wet-strength resins or any other diamine compounds. In one embodiment, plastic-based carriers, such as polyester or polyamide, are prepared and bonded to a substrate. The carriers identified here are merely illustrative of suitable carriers, but may be readily substituted for other carriers by one of ordinary skill in the art. Commercially available filter papers may serve as suitable carriers. Each prepared carrier is bonded to a dye/substrate compound. The carrier/substrate/dye compound may be used as a stand-alone test device or may be secured to a secondary carrier matrix as described below. In another embodiment, multiple carriers are affixed to a single secondary carrier matrix with each carrier being bonded to a different dye-substrate compound for detecting a different chemical or biological agent. In this way, a single test strip could be constructed for individual detection of multiple agents.

The dye/substrate compound is chemically bonded to the carrier by any suitable chemical reaction known in the art. Use of a cross-linker is one mechanism for linking the dye/substrate to the carrier. The cross-linker serves to connect the functional groups of two compounds to one another to form a single compound. One suitable cross-linker is carbodiimide, which serves to link a first compound to a second compound. Carbodiimides are a class of compounds which are known in the art and have various functional groups which are suitable for linking one compound to a second compound. Specific examples of linking a dye/substrate to a carrier are provided in the Examples below.

The test device of the present invention is described herein as being prepared through a standard wet chemistry process where a solution is prepared and impregnated on a carrier which thereby bonds a substrate to the carrier. This description is not meant to be limiting, and it is understood that this disclosure includes other test device preparation techniques, including, but not limited to, printing reagents onto a carrier. Printing techniques for applying reagents to a carrier are known in the art and include such printing methods as screen printing, transfer printing and ink jet printing (see Ink Jet Printing method, EP0202656, incorporated herein by reference).

The secondary carrier matrix is preferably a polymeric material such as polyester or polyamide or other water insoluble resin which is sufficiently durable to withstand the environment in which it is used. Examples of suitable secondary carrier matrices include, but are not limited to, those constructed from polyester or polyamide. The carrier matrix is optionally affixed to the secondary carrier matrix to form the test device. The prepared substrate-bonded carrier matrix is affixed to the secondary carrier matrix with adhesive, hot melt wax, or by other methods commonly known to those skilled in the art. Use of a secondary carrier matrix is optional, but is advantageous where the selected carrier matrix lacks sufficient durability to withstand the harsh environment of the test environment.

When used in a test environment, the test device may be placed loose, or may be secured using a clip or other fastener to hold the device in place. Alternatively, the device is placed in a housing designed to secure the test device. In one embodiment, the housing is designed to partially obstruct a portion of the reactive surface of the test device, mimicking a crevice or panel as would typically be found on a medical instrument. Such housing would provide additional diagnostic feedback to determine how thoroughly the medical instrument washer performs its cleaning functions, and the effectiveness of the detergent in cleaning the crevices of a medical instrument. In another embodiment, the housing allows for the test device to selectively interact with one or more cycles of a medical instrument washer, or with a portion of one or more cycles of the washer. In such a way, the housing is designed to selectively shield the test device during one cycle while exposing the device during another cycle. The housing selectively shields or exposes the carrier in a multitude of ways as are known in the art, such as by opening or closing based on: detecting the presence of moisture, temperature or pressure; use of a timer; receiving a signal from the medical instrument washer; or through manipulation by a technician.

In one embodiment, the carrier or test device includes a control area colored with a dye such that the control area remains unaffected in the presence of the target chemical or biological agent. This control area maintains a constant color during the analysis of a target chemical or biological agent and is used to compare the color change on the reactive portion of the test device, serving as a standard of comparison. In another embodiment, the control area will be on a separate object which is not placed in the test environment.

The test device described herein indicates the presence of a target chemical or biological agent by a color change on the test device. The test device also can be used to indicate the concentration of a chemical or biological agent in a test solution. The chemical or biological agent will typically be one component of a test solution, and the test device of the present invention provides an indication of the concentration of the agent in the solution. As used in a medical instrument washer, a detergent will typically be a portion of a solution which is used to clean medical instruments. This solution will generally at least contain the detergent and a diluting agent, such as water. In another embodiment the solution contains detergent only. The degree of color change on the reactive area of the test device indicates the relative concentration of a detergent in the washer solution. For example, where the test device is initially a first color, a change of color to a second color indicates the presence of the target chemical or biological agent and the degree of color change indicates the concentration of the agent. A technician compares the resulting color on the test device to a legend or key to translate the resulting color to the concentration of the target agent in the test solution.

In another embodiment of the present invention, a water-protective film is used with the test device. Monosol® M1030 is one example of a suitable commercially available water-protective film. The water-protective film is applied over the reactive portion of the test device to shield the test device from reaction with the target chemical or biological agent when exposed to low temperature test solutions. When exposed to elevated temperature detergent solutions, the water-protective film dissolves, thereby allowing the target chemical or biological agent to interact with the reactive portion of the test device. By using a water-protective film, a test device can be designed which preferentially indicates the presence and concentration of an agent in a warm-temperature test solution but remains unaffected in a cool-temperature solution. These water-protective films are commercially available, and the solubility temperature varies by type of film used. An appropriate water-protective film is selected according to the conditions present in a particular test environment. The water-protective film is affixed to the test device according to the manufacturer's instructions or as is known in the art.

In another embodiment, the test device includes an inhibitor which serves to deactivate the reactive portion of the test device from further reaction after the inhibitor has been triggered. The inhibitor may be triggered in many ways, such as by the temperature of the solution, the moisture content on the test device, or the concentration of detergent. Once triggered, the inhibitor serves to prevent the test device from interacting with any surrounding solution, even if the solution contains a target chemical or biological agent. In this way, the inhibitor can be designed to allow a test device to be designed which provides an indicator of the concentration of an agent during a specific part of a test cycle, while remaining unreacted during the balance of the cycle. In a further embodiment, an inhibitor is used in conjunction with a water-protective film such that the test device selectively tests for the presence and concentration of the target agent in a middle part of a test cycle while remaining unreacted during all other parts of the cycle. Suitable inhibitors for the substrates used in the test devices can be chosen from known cross-linkers for the proteins, starches, lipids or cellulose as used in the substrates for the test devices.

The test device of the preferred embodiment includes a dye bound to a substrate, with the substrate chemically bound to a carrier. An alternative embodiment has the carrier affixed to a secondary carrier matrix. Another embodiment has a temperature sensitive film affixed over the dye/substrate on the carrier. An additional embodiment includes a direct dye on the carrier in addition to the dye/substrate. A further embodiment incorporates an inhibitor along with the dye/substrate. Examples of these and other embodiments are provided below.

### Test Device Embodiments

The test device of the present invention may be manifested in various embodiments. What follows is a description of representative embodiments. These representative embodiments and others are described in further detail in the Examples below. These embodiments are merely illustrative of possible embodiments of the present invention, and should not be read as limiting the present invention to the embodiments described.

Amine-Containing Substrate to Carboxyl-Containing Carrier

In one embodiment, an amine-containing substrate, such as a protein, is bound to a carboxyl-containing carrier. The carrier matrix is represented by Formula (1), where R₁ represents the carrier matrix, such as carboxymethyl cellulose or other suitable matrix.

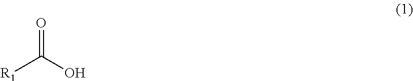

First, a reactive dye is bonded to an amine-containing substrate by methods as are readily known in the art, producing a dye-labeled substrate as represented by Formula (2), where R₄ is a dye-labeled substrate. Alternatively, Formula (2) represents a commercially available dye-labeled substrate.

R₄—NH₂ (2)

Next, a carbodiimide or other suitable cross-linker, such as 1-ethyl-3-(3-dimethylaminopropyl)carbodiimide (EDC) is used to facilitate bonding the carboxyl group of the carrier matrix to the amine group of the dye-labeled substrate. EDC is represented by Formula (3), where R₂ and R₃ are represented in Formulas (4) and (5), respectively. EDC is but one of many carbodiimides that is represented by Formula (3). In another embodiment, R₂ or R₃ are readily replaced by other groups as are known in the art to form other carbodiimides for use in the present reaction.

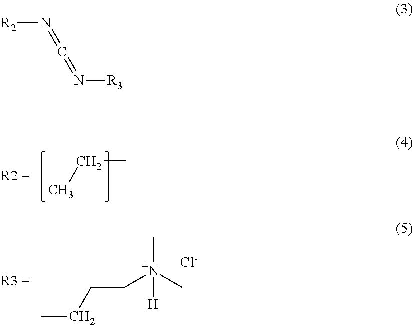

EDC is added to a pH buffered solution containing the dye-labeled substrate of Formula (2) and this solution is then impregnated on the carrier of Formula (1). The EDC facilitates coupling the carboxyl-containing carrier of Formula (1) to the amine-containing dye-labeled substrate of Formula (2), with the target product represented by the amide of Formula (6) where R₁ is the carrier and R₄ is the dye-labeled substrate.

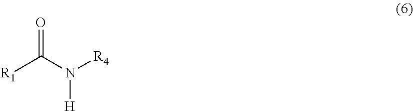

This reaction pathway is illustrated in detail in Example 1, but it should be appreciated by one skilled in the art that the details of that Example may be modified within the scope of this embodiment, such as by substituting for another suitable substrate, carrier or cross-linker to make a test device.

Carboxyl-Containing Substrate to Amine-Containing Carrier

In another embodiment, the test device is formed using a carboxyl-containing substrate, such as a starch, which is bound to an amine-containing carrier. The substrate is bonded to a reactive dye by methods readily known in the art, producing a dye-labeled substrate represented by Formula (7), where R₄ represents a dye-labeled substrate, such as starch or other suitable substrate bonded to a dye.

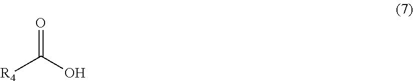

A carrier is chosen suitable for bonding to the dye-labeled substrate of Formula (7). A suitable carrier will include a reactive amine group as shown in Formula (8) where R₁ represents a suitable carrier matrix, such as wool or silk.

R₁—NH₂ (8)

Next, a carbodiimide or other suitable cross-linker, such as EDC is used to facilitate bonding the reactive amine group of the carrier matrix to the carboxyl group of the dye-labeled substrate. EDC is added to a pH buffered solution containing the dye-labeled substrate of Formula (7) and is then impregnated on the carrier of Formula (8). The EDC facilitates coupling the carboxyl-containing dye-labeled substrate of Formula (7) to the amine-containing carrier of Formula (8), with the target product represented by the amide shown in Formula (9) where R₁ represents the carrier and R₄ represents the dye-labeled substrate.

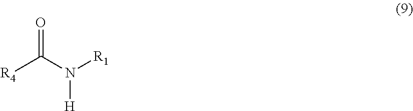

A specific example of this reaction pathway is illustrated in detail in Example 2.

Carboxyl-Containing Substrate to Carboxyl-Containing Carrier

In another embodiment, a soil-like compound is chosen having a carboxyl group which is linked to a carboxyl-containing carrier. The soil-like compound is bonded to a reactive dye by methods as readily known in the art, producing a dye-labeled substrate represented by Formula (10), where R₄ represents a dye-labeled substrate, such as cellulose, starch or other suitable substrate, bonded to a dye.

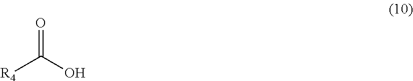

A carrier is chosen, such as cellulose or other carboxyl-containing carrier as represented in Formula (11), where R₁ represents the carrier matrix.

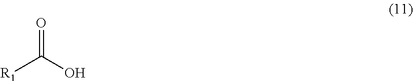

An active amine group is added to the carrier of Formula (11) in a preliminary reaction by cross-linking a diamine to the carboxyl group of the carrier with a carbodiimide or other suitable cross-linker, such as EDC.

A diamine, such as Cadaverine (pentane-1,5-diamine), Putrcine (butane-1,4-diamine) or other diamine as represented by Formula (12), where n is an integer from 1 to 10, preferably 4-6, reacts with EDC or another suitable cross-linker causing one of the amine groups of the diamine to link to the carboxyl group of the carrier resulting in the compound shown in Formula (13), where R₁ represents the carrier matrix.

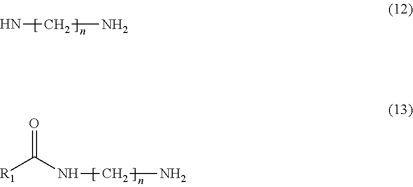

In this way, the carboxyl-containing matrix of Formula (11) is modified to add a reactive amine group and produces the product shown in Formula (13). The primary amine group of Formula (13) is then linked to a carboxyl-containing dye-labeled substrate, such as a starch, as is represented by Formula (10). Here, the reaction is described using a carbodiimide to link a diamine to a carboxyl-containing carrier, though other cross-linkers may be suitable.

Next, EDC or another suitable cross-linker is used to facilitate bonding the amine group of the carrier matrix of Formula (13) to the carboxyl group of the dye-labeled substrate of Formula (10) to form the product shown in Formula (14). This reaction is carried out in a pH buffered solution containing the compounds of Formulas (13) and (10). The EDC forms an intermediate with the carboxyl group of Formula (10) which then reacts with the reactive amine group of formula (13) producing the amide shown in Formula (14).

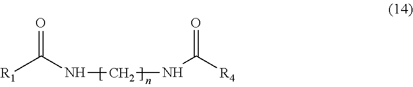

Formula (14) represents a carboxyl-containing carrier, as indicated by R₁ which has been linked to a diamine, which in turn has been bonded to a dye-labeled substrate, as indicated by R₄. In this way, the test device of the present invention is produced using either an amine-containing or carboxyl-containing carrier matrix.

Amine-Containing Substrate to Direct-Dyed Matrix

In another embodiment, the cellulosic carrier is dyed first with a diazo direct dye, as is known in the art, such as Congo Red, Chicago Sky Blue, Direct Orange 31, Direct Violet 51, Direct Yellow 8 or other suitable direct dye. In a second step, the dyed carrier matrix is treated with a cross-linker, as described above, to covalently bond a substrate/dye compound to the dyed carrier. This will result in a mixed-color test device which when exposed to a test solution will react and loose one of the colored components, leaving the other colored component behind. For instance, in one of the Examples described below, a cellulosic carrier matrix is dyed with Congo Red, and then a blue substrate/dye compound having a soil-like substrate is covalently bound to the dyed carrier. The resulting test device has a purple reactive area. Exposure of the test device prepared according to this example to a chemical or biological agent facilitates the cleavage of the substrate causing the blue dye to fall away from the test device causing a color change from purple to red in proportion to the concentration of the agent. Additionally, some direct dyes have indicator properties which may be incorporated into this and other embodiments of the test device as described in more detail below.

Phosphate Containing Substrate to Dyed Carrier Matrix

In another embodiment, a carrier is first dyed with a direct dye such as Congo Red, Chrysoidin, or other direct dye as is known in the art. The direct dye serves as an indicator on the test device. In the preferred embodiment, the direct dye is chosen from a group of indicator dyes known to possess acid-base indicator properties. The dyed-carrier is then treated with a carbodiimide cross-linker, such as Diisopropylcarbodiimide, to covalently bind a phospholipid, such as Dipalmitoyl phosphatidic acid, to the dyed carrier matrix as described above. The resulting test device will employ a protective lipid coating, which prevents interaction between the direct dye and a test solution. The lipid coating reacts and falls away from the test device in the presence of an active lipase-containing agent or a basic hydrolyzing agent. With the lipid coating removed, the direct dye will be exposed to react in acidic medium causing a color change on the test device. In one embodiment, a medical instrument washer will have a first cycle using a basic detergent to hydrolyze any biological materials on a medical instrument and will have a second cycle containing an acidic solution to neutralize the basic detergent. The test device of the present example will react in the basic detergent causing the lipid coating to be removed, and will then react with the acidic solution causing a color change on the direct dye indicator. A color change indicates that both washer cycles functioned properly. Such a test device is readily adaptable for use in other environments where detection of a first basic solution and a second acidic solution is of utility.

In an additional embodiment, a direct dye having a first color, as described above, is dyed on a carrier. In a second step, a dye/phospholipid compound having a second color is prepared and linked to the dyed carrier. This test device shows a mixture of the first color and second color when unreacted. When placed in a medical instrument washer and exposed to a lipase-containing detergent, the dye/phospholipid compound will be removed and cause a color change on the carrier. Exposure to a subsequent acid-wash solution causes another color change on the carrier as the direct dye indicates the presence of the acid. In this way a single test device can be formed which indicates the presence of two agents, and indicates that the agents were presented in a given order.

Inhibitor

In another embodiment, a protein inhibitor is incorporated with the test device. The protein inhibitor serves to deactivate, or impede reaction of the substrate with the target chemical or biological agent. An example of a suitable protein inhibitor is Transglutaminase (TG). TG, and other protein inhibitors, are commercially available, such as from Ajinomoto, and are prepared according to the manufacturer's instructions. The prepared inhibitor is applied in dried form on a secondary carrier. A prepared substrate/dye bound carrier is then applied on top of the inhibitor and affixed to the secondary carrier, whereby the inhibitor is sandwiched between the inhibitor and the carrier. Protein inhibitors are typically temperature activated according to the manufacturer's specifications, though other activation triggers are known in the art. Once activated, the inhibitor serves to prevent reaction of the substrate. One type of inhibitor activates at elevated temperature and cross-links to the dye/substrate, thereby preventing the dye/substrate from falling away from the test device. Thereby, an inhibitor allows selective testing of part of a test cycle and subsequent deactivation of the test device for the remainder of the test.

In an additional embodiment the protein inhibitor is used in combination with a water soluble protective film (as described below) to allow the test device to selectively test a middle cycle of a test and remain unreacted during at least a first and a third test cycle of the test.

Drying Conditions

Following bonding of the dye/substrate to the carrier, the carrier is then optionally dried to remove excess moisture. Drying is performed as is known in the art, such as air-drying or use of an oven or drying tunnel. For example, suitable drying conditions may include placing the prepared carrier in an oven or drying tunnel at a temperature of at least 40° C., but not to exceed 60° C., with a drying duration of at least 7 minutes, but not to exceed 15 minutes. These drying conditions are illustrative of one suitable drying option, and should not be read to limit the invention as one skilled in the art could readily adapt other drying conditions for the present purpose.

Application of Protective Film

In another embodiment, the test device prepared according to the above description is covered with a water protective film, such as MonoSol® M1030 or other similar film as is commercially available. The film serves to prevent a target chemical or biological agent in a test solution from reacting with the test device. The protective film is water soluble at elevated temperatures such that it remains intact when exposed to relatively cool water, such as at 30° C. or below, while it dissolves in the presence of relatively warm water, such as at 50° C. or above. These temperatures are provided for illustrative purposes, the actual temperature of the water solubility of a film will be indicated by the manufacturer or environmental use conditions of the test device. By coating the test device with such a film, the test device can be used to test for the presence of a target agent during a specific step or cycle of the process being tested, such as during a warm-temperature cycle which follows an earlier cool-temperature cycle. This functionality can be helpful as applied to medical instrument washers, because it is common for such washers to have a first cool-temperature prewash cycle, which may contain a detergent, for the gross removal of soil-like material and other debris from the medical instruments. During this first cycle, the reactive portion of the test device remains unreacted. A second heated washer cycle generally follows which uses sufficiently hot-temperature water to dissolve the protective film, thereby exposing the reactive portion of the test device to the detergent in the washer cycle. In this way, a protective film can be used to prepare a test device for selectively indicating the presence or concentration of a target chemical or biological agent in a particular step of a cycle of a test process.

The above-described embodiments represent an abbreviated list of the forms of the test devices of the present invention. These, and other embodiments, are described in further detail in the Examples which follow.

### EXAMPLES

The present invention will be explained concretely with reference to examples as detailed below, the present invention shall not be limited to these examples.

### Example 1

Reactive Orange 16, a reactive dye, is covalently bonded to casein, a protein, according to Wolf (Gerhard A. Wolf; Soluble, dye-labelled substrates for a micro-plate assay of proteinase activity; J. of Microbiol. Methods, 25 (1996) 337-342, incorporated herein by reference). The method described in Wolf produces an aqueous solution of dye-labeled substrate where 84% of the total weight per volume is casein and the balance is Reactive Orange 16.

The dye-labeled substrate produced according to Wolf is covalently bonded to Ahlstrom 642, a cellulose carrier, using EDC dissolved in a buffered dye/substrate solution as follows. 1 mg/ml of EDC is added to a 100 mM phosphate buffer solution at pH 7. The dye/substrate is added to the EDC/phosphate buffer solution to give a concentration of 2.56 mg dye/substrate per ml solution. This solution is then impregnated on the cellulose carrier, thereby bonding the dye/substrate group to the carrier. The carrier is then dried in a 40° C. oven for 10 minutes. The dried carrier is affixed to a polyester secondary carrier matrix with a double-sided adhesive.

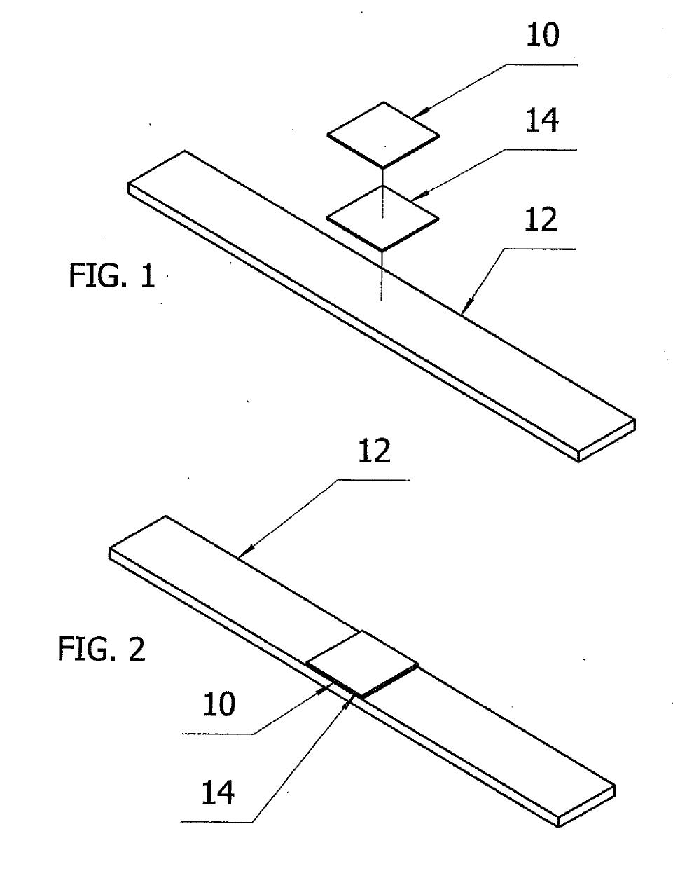

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents a cellulose carrier bonded to dye-labeled casein. FIG. 2 shows the test device of FIG. 1 in assembled form. The shape and configuration of each of the components in FIGS. 1 and 2 is merely illustrative of one embodiment.

The test device prepared according to this Example was exposed to an instrument washer cycle of a medical instrument washer. Several wash cycles were tested, with the concentration of enzyme-containing detergent varying across cycles. Here, the detergent contained Esperase, a subtilisin-type enzyme. The results are summarized in Table 1. The results shown in Table I indicate the two functions of the test device. First, the test device indicates the presence of the active enzyme in the detergent as indicated by any color change on the test device. Second, the degree of color change on the test device correlates with the concentration of enzyme present in the medical instrument washer. These two-fold functions represent significant improvements over the prior art.

TABLE I

| | Unused | Test 1 | Test 2 | Test 3 | Test 4 |
|---|---|---|---|---|---|
| Enzyme Concentration | N/A | 0 | 0.15 | 0.6 | 0.9 |
| (mg/ml) | | | | | |
| Resulting Test Device | Orange/ | Orange/ | Orange | Light | White |
| Color | Red | Red | | orange | |

### Example 2

The test device prepared according to Example 1 was exposed to an instrument cleaning cycle in an ultrasonic cleaner. In a first test, a detergent containing protease was added to the ultrasonic cleaner at 1 oz of detergent per gallon of water at a temperature between 41-43° C. A test device was submerged for 14 minutes in the detergent solution and then allowed to dry. Following submersion, the reactive portion of the test device was observed to be white, indicating the presence of active protease in the detergent. The procedure was repeated in an ultrasonic cleaner using water without detergent. The reactive portion of the test device was observed to be orange, indicating the absence of active protease during the cleaning cycle, as there was no change on the test device. This example illustrates both that the test device of this example is effective in indicating the presence of the test agent, and also that the test device is unaffected by the ultrasonic action of the ultrasonic cleaner.

TABLE II

| | Unused | Test 1 | Test 2 |
|---|---|---|---|
| Detergent Concentration | N/A | 0 | 1 |
| (oz/gal) | | | |
| Resulting Test Device | Orange | Orange | White |
| Color | | | |

### Example 3

Reactive Orange 16 is covalently bonded to carboxymethyl-cellulose according to '421. The method described in '421 produces an aqueous solution of dye-labeled cellulose where 95% of the total weight per volume is carboxymethyl cellulose and the balance is Reactive Orange 16.

The dye-labeled cellulose is covalently bonded to Ahlstrom 973, an amine-containing carrier, using EDC dissolved in a buffered dye/substrate solution as follows. 10 mg/ml of EDC is added to a 100 mM phosphate buffer solution at pH 7. 10 mg/ml of dye-labeled cellulose is added to the phosphate buffer to give a ratio of EDC to dye-labeled cellulose of 1:0.95. The solution is impregnated on the carrier, whereby the substrate is bonded to the carrier. The carrier is then dried in a 40° C. oven for 10 minutes. The dried carrier is then affixed to a polyester secondary matrix with a double-sided adhesive.

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents an amine-containing carrier bonded to dye-labeled cellulose. FIG. 2 shows the test device of FIG. 1 in assembled form. The shape and configuration of each of the components in FIGS. 1 and 2 is merely illustrative of one embodiment.

The test device prepared according to this Example was exposed to an instrument washer cycle of a medical instrument washer. A first test was performed where a washer cycle was run using only water. A second test was performed where an Amylase-containing detergent solution having a concentration of 0.9 mg detergent per ml of solution contacted the test device. The first test caused no color change on the test device, indicating that no enzyme was present in the wash cycle. The second test caused all of the color to be removed from the active area of the test device, indicating the presence of the enzyme at the prescribed concentration. The results of the two tests are summarized in Table III.

TABLE III

| | Unused | Test 1 | Test 2 |
|---|---|---|---|
| Enzyme Concentration | N/A | 0 | 0.9 |
| (mg/ml) | | | |
| Resulting Test Device | Orange | Orange | White |
| Color | | | |

### Example 4

Reactive Orange 16 (RO) is covalently bonded to a carboxyl-containing starch, according to '421. The method described in '421 produces an aqueous solution of dye/substrate where 95% of the total weight per volume is soluble starch and the balance is Reactive Orange 16.

The dye-labeled starch is covalently bonded to Ahlstrom 934, an amine-containing carrier, as follows. 100 mg of dye/substrate, and 100 mg of EDC is added to 50 ml of water for a final concentration of 0.2% dye/substrate and 0.2% EDC. This solution is then impregnated on the carrier, where the EDC facilitates bonding the dye-labeled starch to the carrier matrix. The carrier is then dried in an oven at 40° C. for 10 minutes. The dried carrier is then affixed to a secondary carrier matrix to form a test device.

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents an amine-containing carrier bonded to dye-labeled starch. FIG. 2 shows the test device of FIG. 1 in assembled form. The shape and configuration of each of the components in FIGS. 1 and 2 is merely illustrative of one embodiment.

The test device prepared according to this Example was exposed to an instrument washer cycle of a medical instrument washer. A first test was performed where a washer cycle was run using only water. A second test was performed where an Amylase-containing detergent solution contacted the test device. The first test caused no color change on the test device, indicating that no enzyme was present in the wash cycle. The second test caused all of the color to be removed from the reactive area of the test device, indicating the presence of the enzyme at the prescribed concentration. The results of the two tests are summarized in Table IV.

TABLE IV

| | Unused | Test 1 | Test 2 |
|---|---|---|---|
| Enzyme Concentration | N/A | 0 | 0.6 |
| (mg/ml) | | | |
| Resulting Test Device | Orange | Orange | White |
| Color | | | |

### Example 5

In this example, a test device is prepared using a carboxyl-containing carrier such as Ahlstrom 642 and a carboxyl-containing dye-labeled substrate. To facilitate the use of a carboxyl-containing carrier with a carboxyl-containing dye-labeled substrate, the carrier has an amine group attached thereto. This is accomplished by linking a diamine to the carrier using EDC as follows.

10 mg/ml of EDC and 5 mg/ml of Cadaverine, a diamine, is added to a solution of 100 mM Phosphate Buffer at pH 7. An Ahlstrom 642 carrier is impregnated with the solution, thereby linking the diamine to the carrier. The carrier is then dried in an oven at 40° C. for 10 minutes.

The result of this reaction is a carrier covalently bonded to one of the amine groups of the diamine. The other amine group of the diamine is then bonded to a carboxyl-containing dye-labeled substrate such as cellulose or starch as follows.

A dye-labeled substrate, such as Reactive Orange—Carboxymethyl Cellulose, is prepared according to '421. 10 mg/ml of EDC is added to a 100 mM phosphate buffer solution at pH 7. 10 mg/ml of dye-labeled cellulose is added to the phosphate buffer to give a ratio of EDC to dye-labeled cellulose of 1:0.95. The solution is impregnated on the amine-containing carrier prepared above, whereby the dye-labeled cellulose is bonded to the carrier. The carrier is then dried in a 40° C. oven for 10 minutes. The carrier is then affixed to a polyester secondary carrier matrix using a double sided adhesive to form a test device.

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents a cellulose carrier treated to have a reactive amine group which is then bonded to dye-labeled cellulose. FIG. 2 shows the test device of FIG. 1 in assembled form. The shape and configuration of each of the components in FIGS. 1 and 2 is merely illustrative of one embodiment.

The test device prepared according to this Example was exposed to an instrument washer cycle of a medical instrument washer. A first test was performed with a first test device where a washer cycle was run using only water. A second test was performed with a second test device where a Cellulase-containing detergent solution contacted the test device. The first test caused no color change on the test device, indicating that no enzyme was present in the wash cycle. The second test caused all of the color to be removed from the active area of the test device, indicating the presence of the enzyme at the prescribed concentration. The results of the two tests are summarized in Table V.

TABLE V

| | Unused | Test 1 | Test 2 |
|---|---|---|---|
| Detergent Concentration | N/A | 0 | 0.5 |
| (oz/gal) | | | |
| Resulting Test Device | Orange | Orange | White |
| Color | | | |

### Example 6

In this Example, a carrier such as Ahlstrom 642 was dyed in a first step with Congo Red, a direct diazo-dye. 19.5 mg of Congo Red is added to 30 ml of 0.2M phosphate buffer at pH 6 and 30 ml of water, whereby the dye is incorporated onto the carrier, coloring it red. A Remazol Blue/Casein dye/substrate reagent was prepared as described in Example 1, where Remazol Blue is substituted for Reactive Orange 16. This dye/substrate was cross-linked to the dyed carrier as follows. 216.1 mg of Remazol Blue/Casein and 216.2 mg EDC were dissolved in 25 ml of 0.1M phosphate buffer at pH 6 and 30 ml of water with this solution then impregnated on the carrier. The carrier is then dried at 40° C. for 15 minutes. The dried carrier was then affixed to a polyester secondary carrier matrix. As prepared, the active area of the test device was purple in color.

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents a cellulose carrier which has been dyed with a direct diazo-dye, which then has dye-labeled casein bonded thereto. FIG. 2 shows the test device of FIG. 1 in assembled form. The shape and configuration of each of the components in FIGS. 1 and 2 is merely illustrative of one embodiment.

The test device prepared according to this Example was exposed to an instrument washer cycle. A first test was performed where a first test device was exposed to a washer cycle containing only water. A second test was performed where a protease enzyme (subtilisin type) detergent solution contacted a second test device. The detergent solution was prepared according to the manufacturer's instructions with 0.5 oz detergent per gallon solution. The first test caused no color change on the test device, indicating that no enzyme was present in the wash cycle. The second test caused the blue color to be removed from the active area of the test device changing the color for the test device from purple to red. The presence of the active protease enzyme (subtilisin type) in the detergent is indicated by the color change on the test device. A summary of the results of this example are shown in Table VI.

TABLE VI

| | Unused | Test 1 | Test 2 |
|---|---|---|---|
| Detergent Concentration | N/A | 0 | 0.5 |
| (oz/gal) | | | |
| Resulting Test Device | Purple | Purple | Red |
| Color | | | |

### Example 7

In this Example, an Ahlstrom 642 carrier was dyed in a first step with Trypan blue, a direct diazo-dye as follows. 19.5 mg of Trypan Blue is added to 30 ml of 0.2M phosphate buffer at pH 6 and 30 ml of water, whereby the dye is incorporated onto the carrier. Azocasein, a dye-labeled casein substrate, was cross-linked to the dyed carrier as follows. 1 mg/ml of EDC is added to a 100 mM phosphate buffer at pH 7. The dye/substrate is added to the EDC/phosphate buffer solution to give a concentration of 2.56 mg dye/substrate per ml solution. This solution is then impregnated on the dyed carrier where the EDC facilitates linking the Azocasein to the carrier. The carrier is then dried in a 40° C. oven for 10 minutes. The dried carrier was then affixed to a polyester secondary carrier matrix with double-sided adhesive. As prepared, the reactive area of the test device had a reddish-purple color.

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents a cellulose carrier which has been dyed with a direct diazo-dye, which then has dye-labeled casein bonded thereto. FIG. 2 shows the test device of FIG. 1 in assembled form. The shape and configuration of each of the components in FIGS. 1 and 2 is merely illustrative of one embodiment.

The test device prepared according to this Example was exposed to an instrument washer cycle. A first test was performed where a first test device was exposed to a washer cycle containing only water. A second test was performed where a protease enzyme (subtilisin type) detergent solution contacted a second test device. The detergent solution was prepared according to the manufacturer's instructions with 0.5 oz of detergent per gallon of solution. The first test caused no color change on the test device, indicating that no enzyme was present in the wash cycle. The second test caused the red color to be removed from the active area of the test device changing the color of the test device from reddish-purple to blue. The presence of the active protease enzyme (subtilisin type) in the detergent is indicated by the color change on the test device. A summary of the results of this example are shown in Table VII.

TABLE VII

| | Unused | Test 1 | Test 2 |
|---|---|---|---|
| Detergent Concentration | N/A | 0 | 0.5 |
| (oz/gal) | | | |
| Resulting Test Device | Reddish- | Reddish- | Blue |
| Color | Purple | Purple | |

### Example 8

A test device was prepared by binding a dye containing an amine group to Ahlstrom 642, a carboxyl-containing carrier, using EDC as follows. 25 ml of 0.1 M phosphate buffer at pH 7, 10.1 mg Toluidine Blue 0 and 100.9 mg EDC are combined and impregnated on an Ahlstrom 642 carrier, and then dried at 40° C. for 15 minutes, thereby linking the Toluidine Blue O to the carrier. Then, a Reactive Orange/Casein dye-labeled substrate is covalently bonded to the dyed carrier using EDC dissolved in a buffered dye/substrate solution as follows. 25 ml 0.1 M phosphate buffer at pH 7, 65.8 mg reactive orange-casein and 46.3 mg EDC are impregnated on the prepared Ahlstrom 642 carrier thereby linking the dye-labeled substrate to the dyed carrier. The carrier is then dried at 40° C. for 15 minutes. The dried carrier is then affixed with a double-sided adhesive to a secondary carrier matrix to form a test device.

A representative diagram of this Example is provided in FIGS. 1 and 2. Referring now to FIG. 1, a dye-labeled substrate 10, as prepared according to this Example, is affixed to a secondary carrier 12 by a double-sided adhesive 14. Here, dye-labeled substrate 10 represents a cellulose carrier which has been dyed with a dye having a reactive amine group, dye-labeled casein is then bonded to the reactive dye. FIG. 2 shows the test device of FIG. 1 in assembled form. The shape and configuration of each of the components in FIGS. 1 and 2 is merely illustrative of one embodiment.

A first test device prepared according to this Example was exposed to an instrument washer cycle without a detergent, and the color of the test device did not change. A second test device prepared according to this Example was exposed to an instrument washer cycle containing an active protease enzyme (subtilisin type) detergent. The detergent solution was prepared according to the manufacturer's instructions with 0.5 oz of detergent per gallon of solution. The presence of the detergent was indicated by the color change on the test device from mauve to light blue. The results of this Example are summarized in Table VIII.

TABLE VIII

| | Unused | Test 1 | Test 2 |
|---|---|---|---|
| Detergent Concentration | N/A | 0 | 0.5 |
| (oz/gal) | | | |
| Color of Test Device | Mauve | Mauve | Light Blue |

### Example 9

In this example, an Ahlstrom 642 carrier was dyed with Congo Red direct diazo-dye as follows. A solution of 10 ml water and 8.8 mg Congo Red were impregnated on the carrier and then dried at 50° C. providing a carrier with a red-colored reactive area. Next, a phospholipid was covalently bound to the amine-containing diazo dye using an organic soluble carbodiimide as follows. A solution of 20 ml chloroform/Methanol (2 parts chloroform, 1 part Methanol), 10.6 mg 1,2-Dipalmitoyl-sn-glycero-3-phophate and 200 μl Diisopropylcarbodiimide was prepared and impregnated on the Congo Red-dyed carrier, and then dried at 45° C. The carrier was then affixed to a polyester secondary carrier matrix with a double-sided adhesive to form a test device having a red-colored reactive area and a phospholipid layer.

A representative diagram of this Example is provided in FIGS. 1 and 2. Referring now to FIG. 1, a carrier 10, as prepared according to this Example, is affixed to a secondary carrier 12 by a double-sided adhesive 14. Here, carrier 10 represents a cellulose carrier which has been dyed with an amine-containing direct diazo-dye, with a phopholipid layer bonded to the amine group of the dye. FIG. 2 shows the test device of FIG. 1 in assembled form. The shape and configuration of each of the components in FIGS. 1 and 2 is merely illustrative of one embodiment.

A first test device prepared according to this Example was exposed to a first instrument washer cycle containing a detergent solution containing active lipase enzyme. The detergent was prepared according to the manufacturer's instructions with 0.5 oz of detergent per gallon of solution. The detergent removed the lipid layer from the test device, exposing the Red Congo Dye. The first test device was then exposed to a second instrument washer cycle containing an acid rinse. The acid rinse was Asepti Acid Rinse by Ecolab as prepared according to the manufacturer's instructions to prepare a solution having a pH of 2.5 or below. Here, 0.5 oz of Asepti was used per gallon of acid rinse solution. The first test device was then examined and found to have a blue reactive area.

A second test device prepared according to this Example was exposed to a first instrument washer cycle containing water only, followed by a second cycle containing water only. The second test device was then examined and found to have a red reactive area, indicating no reaction on the reactive area.

A third test device was prepared according to this Example was exposed to a first instrument washer cycle containing a detergent solution containing active lipase enzyme. The test device was then exposed to a second instrument washer cycle containing water only. The third test device was then examined and found to have a Red reactive area, indicating that while the lipid layer had been removed from the test device, the dye did not react in the presence of the second water solution.

The results of the three tests are summarized in Table IX. The results of these tests illustrate that the lipid layer shields the direct dye from reaction with the acid solution. Removing the lipid layer allows the direct dye to change color, indicating the presence of the acid.

TABLE IX

| | Unused | Test 1 | Test 2 | Test 3 |
|---|---|---|---|---|
| Detergent Concentration | N/A | 0.5 | 0 | 0.5 |
| (oz/gal) | | | | |
| Acid Rinse Solution | N/A | 0.5 | 0 | 0 |
| Concentration (oz/gal) | | | | |
| Color of Test Device | Red | Blue | Red | Red |

### Example 10

The test device of Example 1 was prepared and the reactive area was covered with a piece of Monosol M1030 film. Application of protective films to a carrier is well known in the art, here double-sided adhesive strips were applied adjacent opposing edges of the test area of the carrier, and the Monosol film was applied to the adhesive strips, covering the reactive portion of the test device. The film protects the reactive area of the test device from contact with moisture, when the moisture is at low temperatures. At higher temperatures, the film degrades or dissolves and allows the test device to interact with enzymes such as is described in the experimental results of Example 1. By coating the device with the Monosol film, the test device will not react during a cold-temperature phase of a wash cycle even in the presence of a detergent, but will become reactive during a warm-temperature cycle. This allows for a test device which can selectively test one step in the wash cycle independent of other steps which may contain detergent, such as a warm temperature washing cycle, where the warm temperature cycle occurs after a cool temperature cycle.

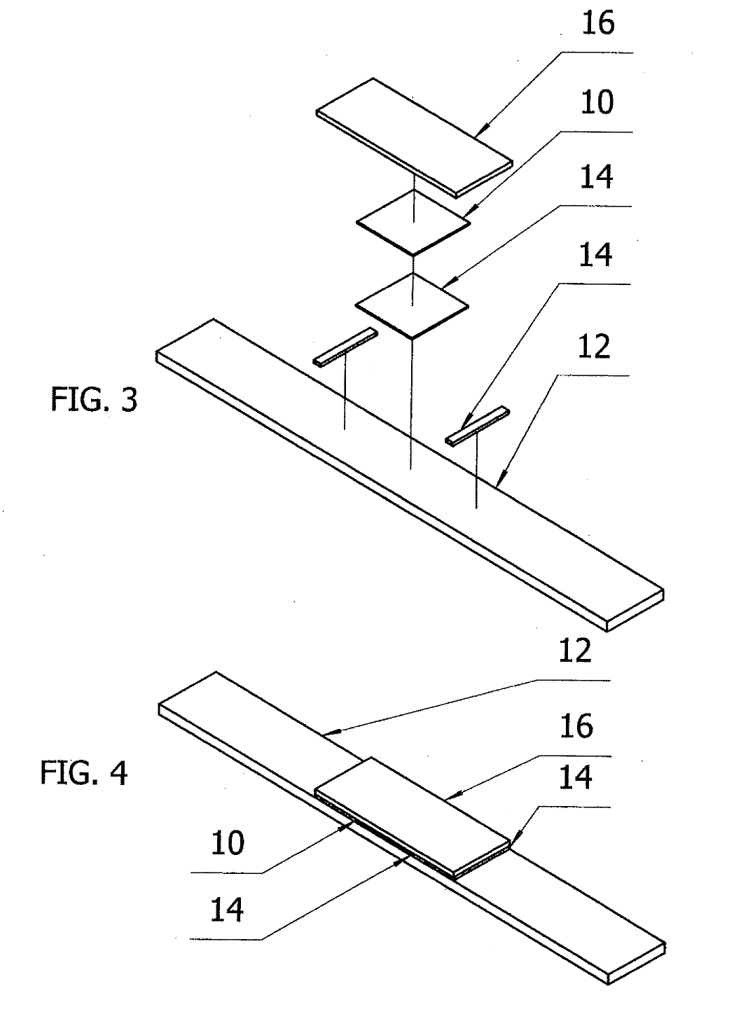

A representative diagram of this Example is provided in FIGS. 3 and 4. Referring now to FIG. 3, a dye-labeled substrate 10, as prepared according to Example 1, is affixed to a secondary carrier 12 by a double-sided adhesive 14. A film 16 is next layered above dye-labeled substrate 10, with the film also affixed to secondary carrier 12 by double-sided adhesive 14. FIG. 3 shows the test device of FIG. 3 in assembled form. The shape and configuration of each of the components in FIGS. 3 and 4 is merely illustrative of one embodiment.

A first test device prepared according to this Example was exposed to an instrument washer cycle of a medical instrument washer using Esperase (subtilisin type) detergent solution as prepared according to the manufacturer's instructions with 0.5 oz of Esperase detergent per gallon of solution, with the detergent solution at 35° C. The resulting color on the test device was recorded as detailed in Table X. A high temperature washer cycle containing a second test device prepared according to this Example was run using the same concentration and type of detergent as above, but at 50° C., and the resulting color on the test device was also recorded. This example illustrates that covering the reactive portion of a test device with a film prevents detergent enzymes from reacting with a test device at low temperatures; at 35° C. the Monosol film remains intact and the enzymes are unable to interact with the reactive area of the test device as evidenced by the lack of color change on the device. At higher temperatures, such as 50° C., the film degrades, thereby exposing the reactive area of the test device to the detergent, with the reactive area of the test device exposed, the enzyme cleaves the substrate, releasing the dye, and causing a color change on the device.

TABLE X

| | Unused | Test 1 | Test 2 |
|---|---|---|---|
| Temperature of Detergent | N/A | 35° C. | 50° C. |
| Detergent Concentration | N/A | 0.5 | 0.5 |
| (oz/gal) | | | |
| Color of Test Device | Orange/Red | Orange/Red | White |

### Example 11

This Example prepares a test device incorporating a carrier and the protein inhibitor Transglutaminase (TG). A test device was prepared according to Example 1, and TG, a protein inhibitor, was added thereto as follows. The TG used in this example was Activa TG (TI), obtained from Ajinomoto. This TG was encapsulated in a hot water soluble carbohydrate coating obtained from Maxx Performance as directed by the manufacturer. The encapsulated TG was applied in dried form on top of a polyester secondary carrier. The test reagent prepared according to Example 1 containing the substrate/dye bound to a cellulose carrier was then applied onto the encapsulated TG with double sided adhesive such that the TG was trapped underneath the carrier, such that the TG was sandwiched between the carrier prepared according to Example 1 and the secondary carrier.

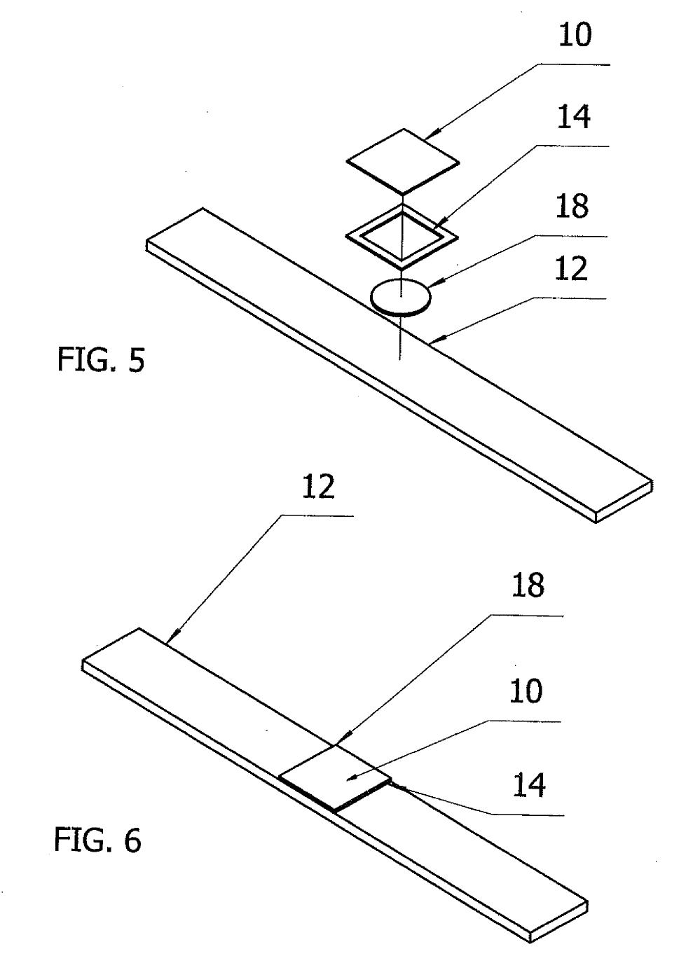

A representative diagram of this Example is provided in FIGS. 5 and 6. Referring now to FIG. 5, an inhibitor 18 is placed on a secondary carrier 12. A dye-labeled substrate 10, as prepared according to Example 1, is affixed to a secondary carrier 12 by a double-sided adhesive 14 over inhibitor 18. FIG. 6 shows the test device of FIG. 5 in assembled form. The shape and configuration of each of the components in FIGS. 5 and 6 is merely illustrative of one embodiment.

A first test device prepared according to this Example was contacted with a protease (subtilisin type) enzyme detergent with the detergent temperature at 50-60° C. The protective encapsulation dissolved in the presence of the elevated-temperature detergent, thereby allowing the inhibitor to prevent interaction between the detergent and the test device. Following the wash cycle, the test device was observed to have an orange coloring, indicating that the test device did not interact with the detergent during the wash cycle. A second test device was prepared according to this Example and was contacted with the protease detergent at a temperature between 35-40° C. Following the wash cycle, the test device was observed to be light orange in color indicating an interaction between the detergent and the test device. In both tests 6 mg of Esperase detergent was present per ml of water in the detergent solution. This Example illustrates the use of an inhibitor to selectively inactivate a test device after a particular occurrence, such as a temperature change, in a test environment. These results are summarized in Table XI.

TABLE XI

| | Unused | Test 1 | Test 2 |
|---|---|---|---|
| Temperature | N/A | Above 40° C. | Below 40° C. |
| Concentration of | N/A | 6 | 6 |
| Esperase (mg/ml) | | | |
| Resulting Color on Test | Orange | Orange | Light Orange |
| Device | | | |

What is claimed is:

1. A test device for detecting the presence of a target agent comprising:
a carrier; and
a dye-labeled substrate chemically bonded to said carrier.

2. The test device as claimed in claim 1, wherein said target agent facilitates the release of said dye from said carrier, thereby causing a color change on said carrier, and indicating the presence of said target agent.

3. The test device as claimed in claim 2, wherein said target agent is an enzyme.

4. The test device as claimed in claim 2, wherein said target agent is a basic solution.

5. The test device as claimed in claim 2, wherein said target agent is a surfactant.

6. The test device as claimed in claim 2, wherein said target agent is in a solution and the degree of said color change on said carrier indicates the concentration of said target agent in said solution.

7. The test device as claimed in claim 2, and a protective film fixed to said carrier and overlying said dye-labeled substrate.

8. The test device as claimed in claim 7, wherein said protective film inhibits interaction between said target agent and said dye-labeled substrate.

9. The test device as claimed in claim 8, wherein said test device is exposed to a first solution at a first predetermined temperature and a second solution at a second predetermined temperature, wherein said protective film dissociates from said carrier when contacted by said second solution.

10. The test device as claimed in claim 1, and said carrier is affixed to a secondary carrier.

11. The test device as claimed in claim 10, wherein said secondary carrier is a polymer.

12. The test device as claimed in claim 1, and an inhibitor contacting said carrier.

13. The test device as claimed in claim 12, wherein said inhibitor is carried between said carrier and a secondary carrier.

14. The test device as claimed in claim 13, wherein said inhibitor reacts to inactivate said test device.

15. The test device as claimed in claim 14, wherein said test device is exposed to a first solution at a first predetermined temperature and a second solution at a second predetermined temperature, wherein said inhibitor reacts in the presence of said second solution.

16. The test device as claimed in claim 15, wherein said inhibitor forms a chemical bond to said substrate when in the presence of said second solution.

17. A test device for detecting the presence of a target agent comprising:
a carrier impregnated with a dye, and a substrate bonded to said carrier, wherein said substrate overlies said carrier

18. The test device as claimed in claim 17, wherein said substrate is hydrophobic.

19. The test device as claimed in claim 17, wherein said substrate is adapted for being cleaved from said carrier in the presence of a first target agent, thereby indicating the presence thereof.

20. The test device as claimed in claim 19, wherein said dye reacts in the presence of a second agent, indicating the presence thereof by a color change on said carrier.
